(19) United States
(12) Patent Application Publication
(10) Pub. No.: US 2018/0244794 A1
(43) Pub. Date: Aug. 30, 2018

(54) ASSESSING AND TREATING LYMPHOMA

(71) Applicant: Mayo Foundation for Medical Education and Research, Rochester, MN (US)

(72) Inventor: Matthew K. Breitenstein, Philadelphia, PA (US)

(73) Assignee: Mayo Foundation for Medical Education and Research, Rochester, MN (US)

(21) Appl. No.: 15/899,601

(22) Filed: Feb. 20, 2018

Related U.S. Application Data
(60) Provisional application No. 62/462,097, filed on Feb. 22, 2017.

(51) Int. Cl. C07K 16/28; A61K 39/395; C12Q 1/6827; A61P 35/00

(52) U.S. Cl. CPC C07K 16/2887; A61K 39/39558; C12Q 1/6827; A61K 31/33; A61P 35/00; A61K 2300/00; C07K 16/2896

(57) ABSTRACT

This document relates to methods and materials involved in assessing and treating mammals (e.g., humans) with non-Hodgkin's lymphoma (e.g., diffuse large B-cell lymphoma). For example, this document provides materials and methods for determining if a human (e.g., a human having non-Hodgkin's lymphoma) has a MAN1A2 variant (e.g., a SNP) that decreases response to treatment (e.g., rituximab treatment). For example, this document provides materials and methods for using one or more alternative non-Hodgkin's lymphoma treatments to treat a mammal having a MAN1A2 variant.

### CROSS-REFERENCE TO RELATED APPLICATIONS

This application claims the benefit of U.S. Provisional Application Ser. No. 62/462,097, filed Feb. 22, 2017. The disclosure of the prior application is considered part of (and is incorporated by reference in) the disclosure of this application.

### STATEMENT REGARDING FEDERAL FUNDING

This invention was made with government support under CA092049 and CA097274 awarded by the National Institutes of Health. The government has certain rights in the invention.

### BACKGROUND

### 1. Technical Field

This document relates to materials and methods involved in assessing and treating mammals (e.g., humans) with non-Hodgkin's lymphoma (e.g., diffuse large B-cell lymphoma). For example, this document provides materials and methods for determining if a human (e.g., a human having non-Hodgkin's lymphoma) has a mannosidase alpha class 1A member 2 (MAN1A2) variant (e.g., a SNP) that decreases response to treatment (e.g., rituximab treatment). For example, this document provides materials and methods for using one or more alternative non-Hodgkin's lymphoma treatments to treat a mammal having a MAN1A2 variant.

### 2. Background Information

Diffuse large B-cell lymphoma (DLBCL) is the most common type of non-Hodgkin's lymphoma (NHL) among adults. Current treatment typically includes R-CHOP (three chemotherapy agents (cyclophosphamide, doxorubicin, and vincristine), and one steroid (prednisone)), which consists of the traditional CHOP, plus rituximab (a monoclonal antibody).

### SUMMARY

This document relates to materials and methods involved in assessing and treating humans with NHL (e.g., DLBCL). For example, this document provides materials and methods for determining if a human having DLBCL has a MAN1A2 variant (e.g., a SNP) that decreases response to treatment (e.g., rituximab treatment).

For the purposes of this document, the term “MAN1A2 variant” refers to a genetic variation (e.g., a substitution, deletion, or insertion of a nucleotide or nucleotides) that is present within a MAN1A2 nucleic acid, results in altered (e.g., increased or decreased) MAN1A2 polypeptide expression, and is associated with a NHL. As described herein, humans having DLBCL with a MAN1A2 variant have a decreased response to rituximab. For example, humans having a single nucleotide polymorphism (SNP; e.g., rs1289877 or r51934249) in MAN1A2 can have a decreased response to rituximab.

Having the ability to determine presence of a MAN1A2 variant can allow clinicians and patients to determine appropriate disease monitoring and care. For example, a human identified as having a MAN1A2 variant as described herein can be selected for treatment with an alternative NHL treatment (e.g., a non-rituximab based treatment such as epratuzumab).

In general, one aspect of this document features a method for assessing response to rituximab. The method includes, or consists essentially of, detecting the presence of a MAN1A2 variant in a biological sample from a mammal, and identifying the mammal as having a decreased response to rituximab based at least in part on the presence of the MAN1A2 variant. The mammal can be a human. The human can have NHL (e.g., DLBCL). The MAN1A2 variant can be SNP (e.g., rs1289877, rs1934249, rs907662, rs1289869, rs1289871, rs10801955, rs1014516, rs1774858, rs1707115, rs1289855, rs1289876, rs1289877, rs1289878, rs1289879, rs1289880, rs946520, rs28648666, rs1146297, rs1289951, rs1289954, rs1146298, rs1146299, rs1289820, rs1289960, rs75814462, rs1289961, rs1009549, rs1679821, rs1289957, rs17037239, rs1289959, rs147796517, rs1316011, rs2256057, rs1289938, rs1289939, rs884748, rs2359248, rs77844631, rs1289947, rs6686707, rs1289948, rs1315646, rs9727433, rs6676578, rs78876272, rs7539143, rs10923293, rs10923294, rs10923297, rs78742938, rs3767797, rs4360510, rs7514323, rs3920835, rs3820500, rs2211130, rs34368125, rs10923316, rs1290539, rs1290541, rs1290542, rs1290543, rs7546624, rs1934249, rs2790033, rs1290549, rs1290551, rs1290552, rs1290553, rs1290556, rs1290557, rs1290558, rs2767318, rs1053902, rs6667122, rs2767320, rs2622362, rs2622363, or rs12738484). The biological sample can include a fluid sample (e.g., blood).

In another aspect of this document features a method for treating NHL in a mammal. The method includes, or consists essentially of, identifying a mammal as having a MAN1A2 variant within a biological sample, and administering an alternative treatment for NHL to the mammal under conditions where the severity of a symptom of non-Hodgkin's lymphoma is reduced. The mammal can be a human. The NHL can be DLBCL. The MAN1A2 variant can be a SNP (e.g., rs1289877, rs1934249, rs907662, rs1289869, rs1289871, rs10801955, rs1014516, rs1774858, rs1707115, rs1289855, rs1289876, rs1289877, rs1289878, rs1289879, rs1289880, rs946520, rs28648666, rs1146297, rs1289951, rs1289954, rs1146298, rs1146299, rs1289820, rs1289960, rs75814462, rs1289961, rs1009549, rs1679821, rs1289957, rs17037239, rs1289959, rs147796517, rs1316011, rs2256057, rs1289938, rs1289939, rs884748, rs2359248, rs77844631, rs1289947, rs6686707, rs1289948, rs1315646, rs9727433, rs6676578, rs78876272, rs7539143, rs10923293, rs10923294, rs10923297, rs78742938, rs3767797, rs4360510, rs7514323, rs3920835, rs3820500, rs2211130, rs34368125, rs10923316, rs1290539, rs1290541, rs1290542, rs1290543, rs7546624, rs1934249, rs2790033, rs1290549, rs1290551, rs1290552, rs1290553, rs1290556, rs1290557, rs1290558, rs2767318, rs1053902, rs6667122, rs2767320, rs2622362, rs2622363, or r512738484). The biological sample can include a fluid sample (e.g., blood). The alternative treatment for NHL can include a non-rituximab therapeutic agent. The non-rituximab therapeutic agent can be cyclophosphamide, aldophosphamide, doxorubicin, vincristine, prednisone, a non-rituximab anti-CD20 antibody (e.g., ublituximab), or an anti-CD22 antibody (e.g., epratuzumab). The alternative treatment for NHL can include a non-chemotherapeutic treatment. The non-chemotherapeutic treatment can be radiation therapy, immunotherapy, or a stem cell transplant.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains a graph showing SNP variants within the MANA1A2 gene region as predicting refractory response (e.g. non-response or poor response) in DLBCL patients treated with R-CHOP.

FIG. 2 contains graphs showing proliferation of lymphoblastoid (LCL) cells (top) and Raji cells (bottom) in the presence of short interfering (si) RNAs targeting MAN1A2, R-CHOP, or both.

FIG. 3 contains graphs showing CHOP cytotoxicity in LCL cells (top) and Raji cells (bottom) treated with siRNAs targeting MAN1A2.

FIGS. 4A-B contain graphs showing rituximab cytotoxicity in general cell line model systems (A) and in DLBCL-specific cell line model systems (B). A) LCL cells (A.1) and Raji cells (A.2) treated with siRNAs targeting MAN1A2 (as used in FIGS. 2 and 3). B) DLBCL-specific cell line model systems treated with siRNAs targeting MAN1A2.

FIG. 5 contains a graph showing that MAN1A2 is expressed in many tissue types relevant to DLBCL, including lymphocytes.

FIGS. 6A-B contain expression quantitative trait locus (eQTL) graphs displaying the impact of variant rs1934249 on MAN1A2 gene expression within select tissues. Variant rs1934249 (A) decreased MAN1A2 gene expression in skin (sun exposed and non-sun exposed), muscle, and esophageal tissues. Variant rs1934249 (B) increased MAN1A2 gene expression in fibroblast model systems.

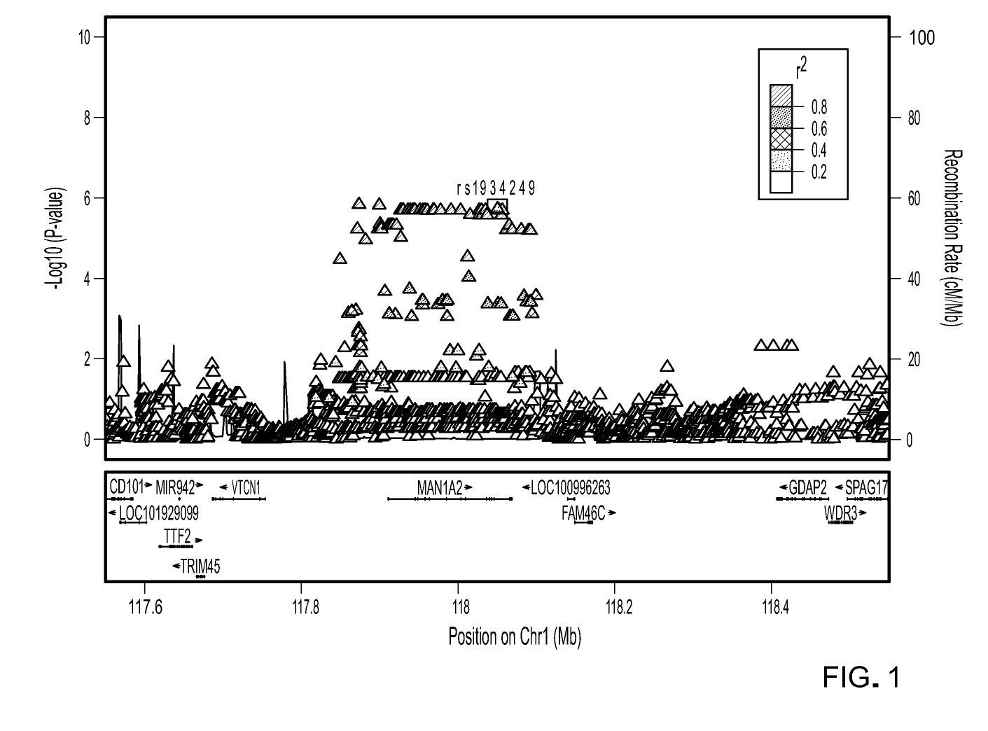

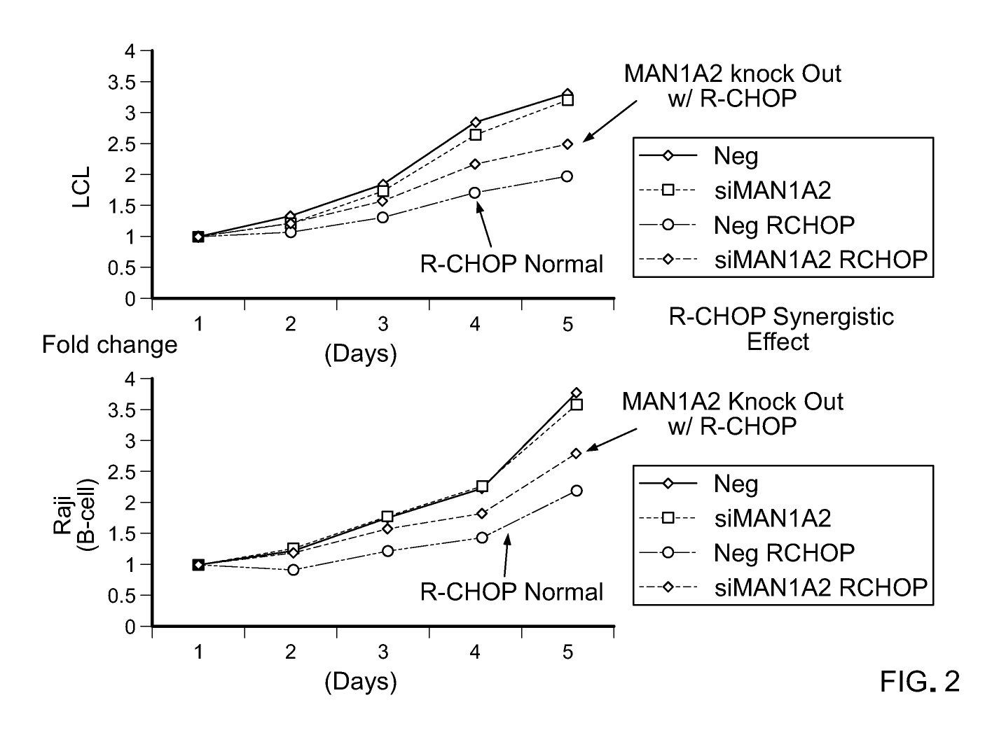

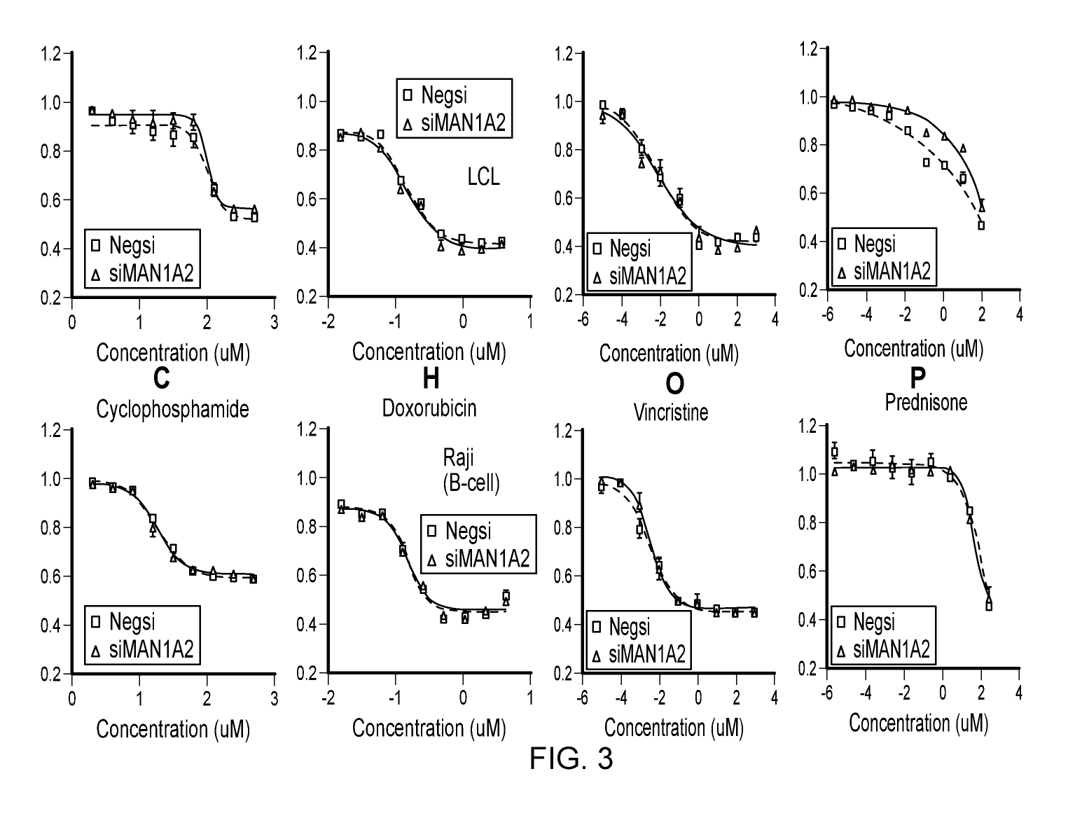

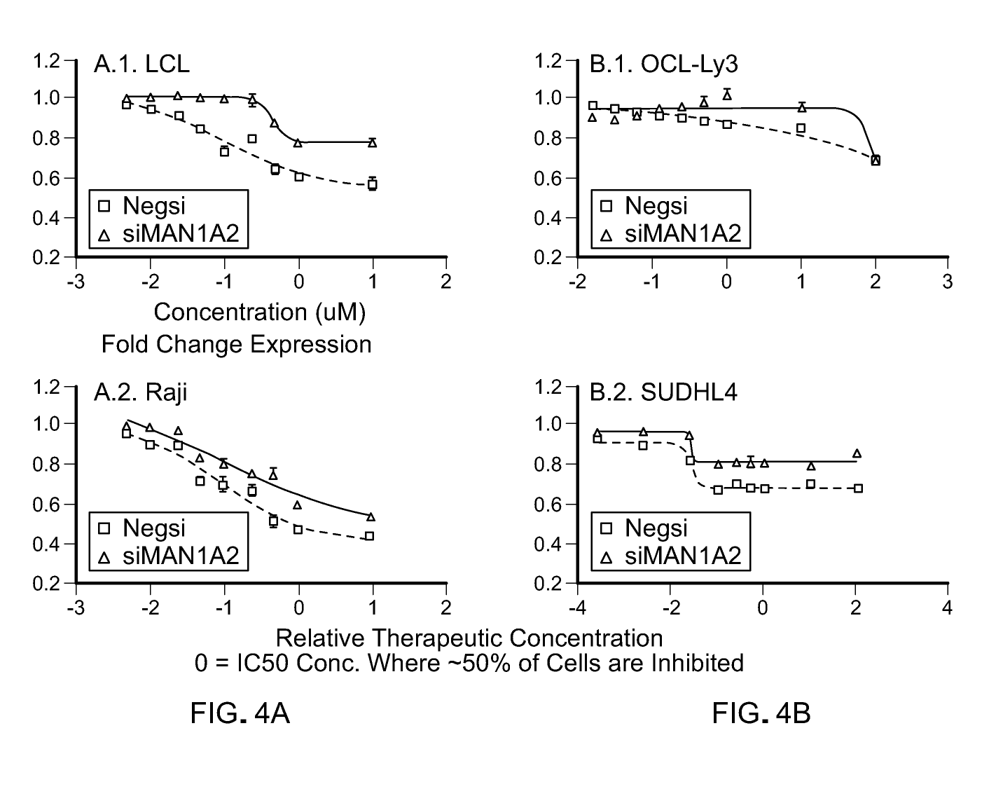

### DETAILED DESCRIPTION

This document provides materials and methods for assessing humans having or suspected of having NHL. As described herein, the presence of a MAN1A2 variant (e.g., a SNP) can indicate that a mammal (e.g., a human) having NHL may have a decreased response to rituximab (e.g., R-CHOP) treatment. For example, a mammal having a MAN1A2 SNP (e.g., rs1289877 or r51934249) can have a decreased response to rituximab. This document also provides materials and methods for treating mammals with NHL. For example, this document provides methods and materials for using one or more alternative treatments for NHL, to reduce the proliferation of lymphoma cells, and/or to reduce the number of lymphoma cells within a mammal. In some cases, one or more alternative treatments can be administered to a human having NHL where the human has a SNP (e.g., rs1289877 or r51934249) in MAN1A2.

Examples of MAN1A2 variants include, without limitation, those listed in Table 1. A MAN1A2 variant can be any genetic variant present within a MAN1A2 nucleic acid that causes decreased response to rituximab. A MAN1A2 variant can be in a coding region of MAN1A2, in a non-coding region of MAN1A2, or in an intergenic region. A MAN1A2 variant can be a SNP. Examples of MAN1A2 variants include, without limitation, the rs1289877 SNP and the rs1934249 SNP. In cases where a MAN1A2 variant is a SNP, the SNP can be biallelic.

TABLE 1
MAN1A2 variants

| | | | Variant | WT (major) |
|---|---|---|---|---|
| SNP | Chromosome | Base Pair | (minor) Allele | Allele |
| rs1289877 | 1 | 117898944 | T | C |
| rs1934249 | 1 | 118049831 | T | A |
| rs1289960 | 1 | 117926118 | C | T |
| rs1289938 | 1 | 117944341 | G | A |
| rs7539143 | 1 | 117977324 | G | A |
| rs34368125 | 1 | 118026538 | A | C |
| rs1290549 | 1 | 118051748 | G | C |
| rs1290553 | 1 | 118056073 | T | G |
| rs1315646 | 1 | 117960377 | A | G |
| rs1009549 | 1 | 117927803 | T | G |
| rs1679821 | 1 | 117929605 | G | A |
| rs1289957 | 1 | 117935635 | A | T |
| rs1289959 | 1 | 117938741 | A | G |
| rs1316011 | 1 | 117941924 | C | T |
| rs2256057 | 1 | 117942591 | T | C |
| rs1289939 | 1 | 117944435 | T | C |
| rs884748 | 1 | 117950847 | A | G |
| rs1289947 | 1 | 117954586 | T | G |
| rs1289948 | 1 | 117957288 | A | G |
| rs6676578 | 1 | 117968957 | G | A |
| rs4360510 | 1 | 117990539 | A | G |
| rs1290539 | 1 | 118029601 | T | G |
| rs1290542 | 1 | 118034495 | A | G |
| rs7514323 | 1 | 118002719 | A | G |
| rs9727433 | 1 | 117967602 | T | A |
| rs2211130 | 1 | 118014894 | C | T |
| rs10923316 | 1 | 118026778 | C | T |
| rs1290541 | 1 | 118030256 | T | G |
| rs1290543 | 1 | 118036420 | G | A |
| rs1290552 | 1 | 118054772 | G | T |
| rs1289878 | 1 | 117900155 | A | T |
| rs1289879 | 1 | 117900918 | A | G |
| rs1146297 | 1 | 117910682 | G | A |
| rs1289951 | 1 | 117913019 | G | A |
| rs1289954 | 1 | 117914309 | G | A |
| rs1146299 | 1 | 117916880 | G | C |
| rs1289820 | 1 | 117921039 | T | G |
| rs1290557 | 1 | 118064763 | T | C |
| rs1289880 | 1 | 117900925 | G | T |
| rs1146298 | 1 | 117915907 | C | T |
| rs946520 | 1 | 117901462 | G | A |
| rs1290556 | 1 | 118061381 | T | A |
| rs2767318 | 1 | 118068558 | A | G |
| rs2767320 | 1 | 118080220 | G | A |
| rs2622362 | 1 | 118089166 | G | T |
| rs2622363 | 1 | 118089341 | C | T |
| rs12738484 | 1 | 118091945 | G | C |
| rs1289961 | 1 | 117927337 | G | A |
| rs1707115 | 1 | 117873222 | C | T |
| rs1774858 | 1 | 117873022 | C | G |
| rs1289876 | 1 | 117897700 | G | A |
| rs1014516 | 1 | 117871130 | C | T |
| rs1289855 | 1 | 117881607 | C | T |
| rs3920835 | 1 | 118011751 | T | C |
| rs907662 | 1 | 117848822 | A | G |
| rs75814462 | 1 | 117926372 | T | A |
| rs3820500 | 1 | 118013322 | G | A |
| rs1289869 | 1 | 117859527 | C | T |

Any appropriate sample can be assessed to determine if a mammal (e.g., a human) has a MAN1A2 variant. For example, biological samples such as fluid samples (e.g., blood, serum, or plasma) can be obtained from a human and assessed for the presence of a MAN1A2 variant. In some cases, a blood sample can be obtained and assessed to determine whether or not the human has a MAN1A2 variant.

Any appropriate method can be used to detect the presence or absence of a MAN1A2 variant (e.g., a SNP) within a sample (e.g., a blood sample). For example, methods of detecting the presence or absence of a SNP include, without limitation, sequencing (e.g., next generation sequencing), mass spectrometry, single-strand conformation polymorphism (SSCP), and restriction fragment length polymorphism (RFLP). In some cases, genomic DNA can be sequenced to determine the presence or absence of a MAN1A2 variant. In some cases, cDNA generated from MAN1A2 mRNA (e.g., MAN1A2 cDNA generated by RT-PCR) can be sequenced to determine the presence or absence of a MAN1A2 variant.

Once identified as having a MAN1A2 variant (e.g., a SNP), a mammal (e.g., a human) can be administered or instructed to self-administer any appropriate alternative treatments for NHL. As used herein an alternative NHL treatment can be any non-rituximab therapeutic agent or any other treatment procedure. Examples of non-rituximab therapeutic agents that can be used to treat NHL include, without limitation, cyclophosphamide, aldophosphamide, doxorubicin, vincristine, prednisone, non-rituximab anti-CD20 antibodies (e.g., ublituximab), and anti-CD22 antibodies (e.g., epratuzumab). Examples other treatment procedures that can be used to treat NHL include, without limitation, radiation therapy, immunotherapy (e.g., radioimmunotherapy), and stem cell transplants. For example, when a mammal (e.g., a human) having NHL is identified as having MAN1A2 variant (e.g., rs1289877 or rs1934249), the mammal can be administered CHOP (e.g., cyclophosphamide, doxorubicin, vincristine, and prednisone). For example, when a mammal (e.g., a human) having NHL is identified as having MAN1A2 variant (e.g., rs1289877 or r51934249), the mammal can be administered a non-rituximab anti-CD20 antibody and/or an anti-CD22 antibody. When a mammal (e.g., a human) is identified as having a non-variant MAN1A2 (e.g., having a wild type SNP allele), the mammal can be administered or instructed to self-administer rituximab (e.g., R-CHOP).

In some cases, the materials and methods provided herein can be used to reduce the symptoms of NHL. Symptoms of NHL can include, without limitation, swollen lymph nodes (e.g., in your neck, armpits or groin), abdominal pain or swelling, chest pain, coughing or trouble breathing, fatigue, fever, night sweats, and weight loss.

When treating NHL as described herein, the NHL can be any appropriate NHL. Examples of NHLs that can be treated as described herein include, without limitation, DLBCL and follicular lymphoma, mantle cell lymphoma, small lymphocytic lymphoma, and CD20-positive NHLs. For example, the NHL treated as described herein can be DLBCL. In some cases, the number of lymphoma cells present within a mammal (e.g., a human) can be reduced using the materials and methods described herein.

Any type of mammal having NHL can be treated as described herein. For example, humans and other primates such as monkeys having NHL and a MAN1A2 variant (e.g., a SNP) can be treated with one or more alternative treatment for NHL as described herein. In some cases, dogs, cats, horses, cows, pigs, sheep, rabbits, mice, and rats can be treated with one or more alternative treatment for NHL as described herein.

The invention will be further described in the following examples, which do not limit the scope of the invention described in the claims.

### Examples

### Example 1: MANA1A2 Expression in DLBCL

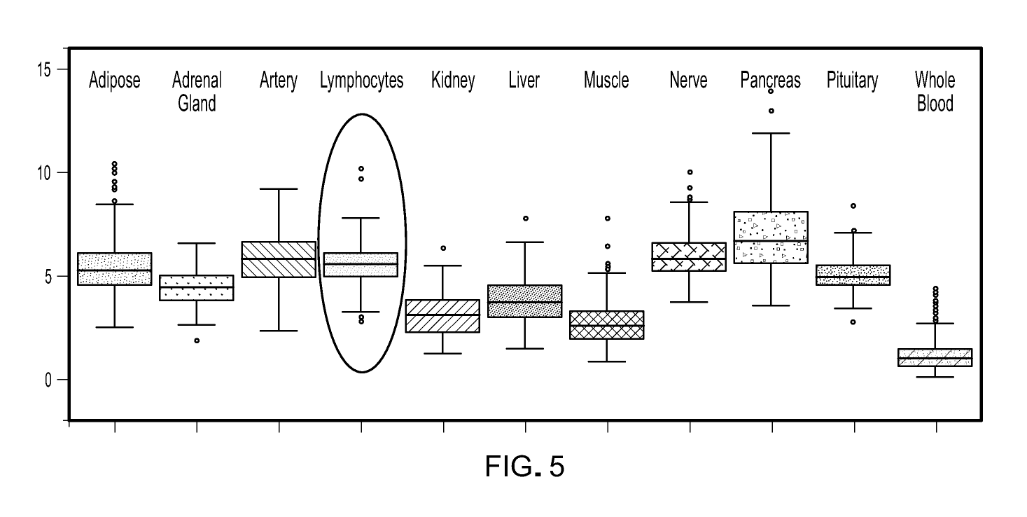

MAN1A2 is a protein known to be expressed in tissue types relevant to DLBCL (FIG. 5).

### Example 2: MAN1A2 Targets Rituximab

Cell proliferation experiments were performed on cell lines having MANA1A2 expression (e.g., LCL cells and Raji B-cells) to evaluate sensitivity to therapeutic agent exposure in comparison with MAN1A2 knock down. Pharmacologic studies were sought to confirm the MAN1A2 gene candidate and demonstrate efficacy on the individual agent Rituximab and the combination R-CHOP regimen. Pharmacologic studies were performed on cell line model systems. Cytoxocity experiments that compare the ability of therapeutic exposures and biologic manipulation in combination to kill a percentage of cells were utilized to demonstrate efficacy efficacy of therapeutic agents in vitro. Anti-CD20 (Rituximab), Chimeric Antibody and MAN1A2 siRNA (h) were the primary assays utilized for in vitro experimentation.

In vitro characterization [TRL 3] determined MAN1A2 to be responsible for sensitivity to 1) Rituximab as a single agent in lymphoblastoid, Raji, OCI-Ly3, and SUDHL4 cell line models, and 2) Rituximab in combination with the clinical standard CHOP (C—Cyclophosphamide; H—Doxorubicin; O—Vincristine; P—Prednisone) regimen lymphoblastoid and Raji cell line models. Specifically, MAN1A2 knock downs demonstrated strong decreased response to Rituximab (˜25%) as a single agent and in combination R-CHOP therapy (˜20%) in both general and DLBCL specific cell line model systems.

### Example 3: MAN1A2 Variants Cause Decreased Response to Rituximab

### Methods

Potential gene candidates indicative of treatment response to R-CHOP were generated. Newly diagnosed DLBCL patients treated with R-CHOP, and with germline DNA and genotyping data were selected. Refractory disease was defined as either i) a best response of stable or progressive disease, or ii) progressive disease by the end of the planned number of cycles of R-CHOP. Odds ratios (ORs) and 95% confidence intervals (CI) were used to estimate the association of SNPs with refractory disease, controlling for age, gender, and age-adjusted International Prognostic Index. An independent cohort using similar criteria and based on a comparable patient population was utilized to replicate potential associations. Pooled associations were utilized to demonstrate statistical significance and effect size.

### Results

A series of results indicated the efficacy of MAN1A2 variants as prognostic of non-response to Rituximab. One DLBCL patient cohort was treated with R-CHOP. Patient cohort characteristics shown in the table below. Patient responses to R-CHOP were evaluated using standard clinical assessment methods. Approximately 10% of DLBCL patients were refractory to R-CHOP.

Mayo Clinic DLBCL Cases

| | | R-CHOP | | |
|---|---|---|---|---|
| | Non Refractory | Refractory | Total | |
| | (N = 424) | (N = 40) | (N = 464) | p value |
| Diagnosis | | | | |
| Age | | | | |
| Mean | 59.3 | 59.8 | 59.4 | 0.93411 |
| Range | (18.0-92.0) | (20.0-84.0) | (18.0-92.0) | |
| Gender | | | | |
| F | 198 (46.7%) | 14 (35.0%) | 212 (45.7%) | 0.15572 |
| M | 226 (53.3%) | 26 (65.0%) | 252 (54.3%) | |
| Age Group | | | | |
| <=60 | 200 (47.2%) | 16 (40.0%) | 216 (46.6%) | 0.38482 |
| >60 | 224 (52.8%) | 24 (60.0%) | 248 (53.4%) | |
| IPI | | | | |
| 0-1 Low | 264 (62.3%) | 12 (30.0%) | 276 (59.5%) | 0.00032 |
| 2 Low | 123 (29.0%) | 20 (50.0%) | 143 (30.8%) | |
| Intermediate | | | | |
| 3 High | 37 (8.7%) | 8 (20.0%) | 45 (9.7%) | |
| Intermediate | | | | |

¹Kruskal Wallis
²Chi-Square

The median age at diagnosis of 424 of DLBCL patients treated with R-CHOP was 62 years (range 18-92); 45.7% were female; and 58.4% had Ann Arbor Stage By the end of initial therapy 40 patients had primary refractory disease. Of the 1,040 SNPs evaluated, 120 (12%) were significantly associated with refractory disease at the 5% significance threshold. SNP variant rs1934249 MAN1A2 was associated with clinical non-response (e.g. was refractory) to R-CHOP therapy in DLBCL patients with statistical significance (p=1.47e-06) and large effect (OR=3.58). Associations of tagging SNPs were identified across the MAN1A2 gene as meeting the Technology Readiness Level for BioPharma (TRL) 1 and 2 of predicting response to R-CHOP therapy.

Variant SNPs evaluated within the MAN1A2 gene on chromosome 1 were identified to have similar statistical significance with large effect size. Evaluated MANA1A2 SNPs included: rs907662, rs1289869, rs1289871, rs10801955, rs1014516, rs1774858, rs1707115, rs1289855, rs1289876, rs1289877, rs1289878, rs1289879, rs1289880, rs946520, rs28648666, rs1146297, rs1289951, rs1289954, rs1146298, rs1146299, rs1289820, rs1289960, rs75814462, rs1289961, rs1009549, rs1679821, rs1289957, rs17037239, rs1289959, rs147796517, rs1316011, rs2256057, rs1289938, rs1289939, rs884748, rs2359248, rs77844631, rs1289947, rs6686707, rs1289948, rs1315646, rs9727433, rs6676578, rs78876272, rs7539143, rs10923293, rs10923294, rs10923297, rs78742938, rs3767797, rs4360510, rs7514323, rs3920835, rs3820500, rs2211130, rs34368125, rs10923316, rs1290539, rs1290541, rs1290542, rs1290543, rs7546624, rs1934249, rs2790033, rs1290549, rs1290551, rs1290552, rs1290553, rs1290556, rs1290557, rs1290558, rs2767318, rs1053902, rs6667122, rs2767320, rs2622362, rs2622363, and rs12738484. These results indicated a strong, robust signal that variant SNPs within the MAN1A2 gene alters the MAN1A2 gene product to one that becomes clinically non-responsive to Rituximab.

### Example 4: SNP and MAN1A2 Gene Expression

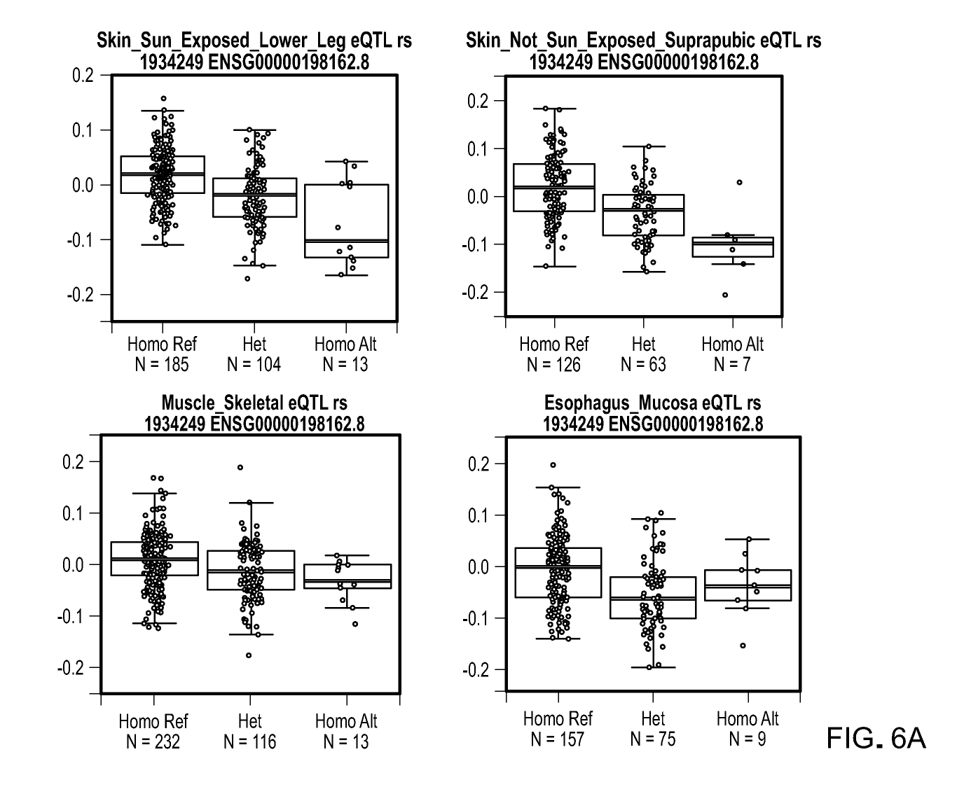

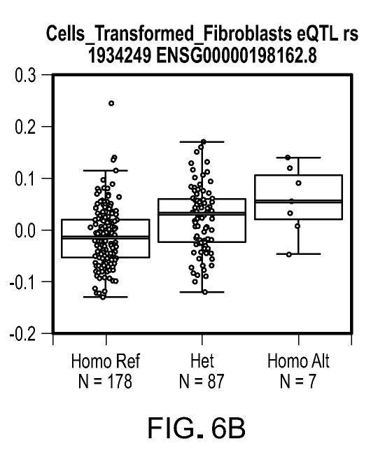

To evaluate the effect of MAN1A2 variants (e.g., SNPs), expression quantitative trait locus (eQTL) was performed. As shown in FIG. 6A, variant rs1934249 resulted in increased MAN1A2 gene expression within select tissues (e.g., skin (sun exposed and non-sun exposed), muscle, and esophageal tissues). However, as shown in FIG. 6B, variant rs1934249 increased MAN1A2 gene expression in fibroblast model systems. In some cases, immunohistochemistry (IHC) techniques, mass spectrometry techniques (e.g., proteomics-based mass spectrometry assays or targeted quantification-based mass spectrometry assays), western blotting techniques, and quantitative RT-PCR techniques can be used to determine whether or not a MAN1A2 variant (e.g., a SNP) alters MAN1A2 expression.

### Example 5: Treating DLBCL

A biological sample (e.g., blood) is obtained from a human having DLBCL. The obtained sample is examined for the presence or absence of a MAN1A2 SNP set forth in Table 1 (e.g., a rs1289877 SNP, a rs1934249 SNP, a rs907662 SNP, a rs1289869 SNP, a rs1289871 SNP, a rs10801955 SNP, a rs1014516 SNP, a rs1774858 SNP, a rs1707115 SNP, a rs1289855 SNP, a rs1289876 SNP, a rs1289877 SNP, a rs1289878 SNP, a rs1289879 SNP, a rs1289880 SNP, a rs946520 SNP, a rs28648666 SNP, a rs1146297 SNP, a rs1289951 SNP, a rs1289954 SNP, a rs1146298 SNP, a rs1146299 SNP, a rs1289820 SNP, a rs1289960 SNP, a rs75814462 SNP, a rs1289961 SNP, a rs1009549 SNP, a rs1679821 SNP, a rs1289957 SNP, a rs17037239 SNP, a rs1289959 SNP, a rs147796517 SNP, a rs1316011 SNP, a rs2256057 SNP, a rs1289938 SNP, a rs1289939 SNP, a rs884748 SNP, a rs2359248 SNP, a rs77844631 SNP, a rs1289947 SNP, a rs6686707 SNP, a rs1289948 SNP, a rs1315646 SNP, a rs9727433 SNP, a rs6676578 SNP, a rs78876272 SNP, a rs7539143 SNP, a rs10923293 SNP, a rs10923294 SNP, a rs10923297 SNP, a rs78742938 SNP, a rs3767797 SNP, a rs4360510 SNP, a rs7514323 SNP, a rs3920835 SNP, a rs3820500 SNP, a rs2211130 SNP, a rs34368125 SNP, a rs10923316 SNP, a rs1290539 SNP, a rs1290541 SNP, a rs1290542 SNP, a rs1290543 SNP, a rs7546624 SNP, a rs1934249 SNP, a rs2790033 SNP, a rs1290549 SNP, a rs1290551 SNP, a rs1290552 SNP, a rs1290553 SNP, a rs1290556 SNP, a rs1290557 SNP, a rs1290558 SNP, a rs2767318 SNP, a rs1053902 SNP, a rs6667122 SNP, a rs2767320 SNP, a rs2622362 SNP, a rs2622363 SNP, or a rs12738484 SNP). If a SNP is detected in the sample, then the human is administered a non-rituximab therapeutic agent (e.g., ublituximab and/or epratuzumab). The administered non-rituximab therapeutic can reduce the severity of one or more symptoms of DLBCL.

### Example 6: Treating DLBCL

A human having DLBCL, and identified as having a MAN1A2 SNP set forth in Table 1 (e.g., a rs1289877 SNP, a rs1934249 SNP, a rs907662 SNP, a rs1289869 SNP, a rs1289871 SNP, a rs10801955 SNP, a rs1014516 SNP, a rs1774858 SNP, a rs1707115 SNP, a rs1289855 SNP, a rs1289876 SNP, a rs1289877 SNP, a rs1289878 SNP, a rs1289879 SNP, a rs1289880 SNP, a rs946520 SNP, a rs28648666 SNP, a rs1146297 SNP, a rs1289951 SNP, a rs1289954 SNP, a rs1146298 SNP, a rs1146299 SNP, a rs1289820 SNP, a rs1289960 SNP, a rs75814462 SNP, a rs1289961 SNP, a rs1009549 SNP, a rs1679821 SNP, a rs1289957 SNP, a rs17037239 SNP, a rs1289959 SNP, a rs147796517 SNP, a rs1316011 SNP, a rs2256057 SNP, a rs1289938 SNP, a rs1289939 SNP, a rs884748 SNP, a rs2359248 SNP, a rs77844631 SNP, a rs1289947 SNP, a rs6686707 SNP, a rs1289948 SNP, a rs1315646 SNP, a rs9727433 SNP, a rs6676578 SNP, a rs78876272 SNP, a rs7539143 SNP, a rs10923293 SNP, a rs10923294 SNP, a rs10923297 SNP, a rs78742938 SNP, a rs3767797 SNP, a rs4360510 SNP, a rs7514323 SNP, a rs3920835 SNP, a rs3820500 SNP, a rs2211130 SNP, a rs34368125 SNP, a rs10923316 SNP, a rs1290539 SNP, a rs1290541 SNP, a rs1290542 SNP, a rs1290543 SNP, a rs7546624 SNP, a rs1934249 SNP, a rs2790033 SNP, a rs1290549 SNP, a rs1290551 SNP, a rs1290552 SNP, a rs1290553 SNP, a rs1290556 SNP, a rs1290557 SNP, a rs1290558 SNP, a rs2767318 SNP, a rs1053902 SNP, a rs6667122 SNP, a rs2767320 SNP, a rs2622362 SNP, a rs2622363 SNP, or a rs12738484 SNP) is administered a non-rituximab therapeutic agent (e.g., ublituximab and/or epratuzumab). The administered non-rituximab therapeutic can reduce the severity of one or more symptoms of DLBCL.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing response to rituximab, wherein said method comprises:
detecting the presence of a mannosidase alpha class 1A member 2 (MAN1A2) variant in a biological sample from a mammal; and
identifying said mammal as having a decreased response to rituximab based at least in part on said presence of said MAN1A2 variant.

2. The method of claim 1, wherein said mammal is a human.

3. The method of claim 2, wherein said human has non-Hodgkin's lymphoma.

4. The method of claim 3, wherein said non-Hodgkin's lymphoma is diffuse large B-cell lymphoma.

5. The method of claim 1, wherein said MAN1A2 variant is a single nucleotide polymorphism (SNP).

6. The method of claim 5, wherein said SNP is selected from the group consisting of a rs1289877 SNP, a rs1934249 SNP, a rs907662 SNP, a rs1289869 SNP, a rs1289871 SNP, a rs10801955 SNP, a rs1014516 SNP, a rs1774858 SNP, a rs1707115 SNP, a rs1289855 SNP, a rs1289876 SNP, a rs1289877 SNP, a rs1289878 SNP, a rs1289879 SNP, a rs1289880 SNP, a rs946520 SNP, a rs28648666 SNP, a rs1146297 SNP, a rs1289951 SNP, a rs1289954 SNP, a rs1146298 SNP, a rs1146299 SNP, a rs1289820 SNP, a rs1289960 SNP, a rs75814462 SNP, a rs1289961 SNP, a rs1009549 SNP, a rs1679821 SNP, a rs1289957 SNP, a rs17037239 SNP, a rs1289959 SNP, a rs147796517 SNP, a rs1316011 SNP, a rs2256057 SNP, a rs1289938 SNP, a rs1289939 SNP, a rs884748 SNP, a rs2359248 SNP, a rs77844631 SNP, a rs1289947 SNP, a rs6686707 SNP, a rs1289948 SNP, a rs1315646 SNP, a rs9727433 SNP, a rs6676578 SNP, a rs78876272 SNP, a rs7539143 SNP, a rs10923293 SNP, a rs10923294 SNP, a rs10923297 SNP, a rs78742938 SNP, a rs3767797 SNP, a rs4360510 SNP, a rs7514323 SNP, a rs3920835 SNP, a rs3820500 SNP, a rs2211130 SNP, a rs34368125 SNP, a rs10923316 SNP, a rs1290539 SNP, a rs1290541 SNP, a rs1290542 SNP, a rs1290543 SNP, a rs7546624 SNP, a rs1934249 SNP, a rs2790033 SNP, a rs1290549 SNP, a rs1290551 SNP, a rs1290552 SNP, a rs1290553 SNP, a rs1290556 SNP, a rs1290557 SNP, a rs1290558 SNP, a rs2767318 SNP, a rs1053902 SNP, a rs6667122 SNP, a rs2767320 SNP, a rs2622362 SNP, a rs2622363 SNP, and a rs12738484 SNP.

7. The method of claim 1, wherein said biological sample comprises a fluid sample.

8. The method of claim 7, wherein said fluid sample is blood.

9. A method for treating non-Hodgkin's lymphoma in a mammal, said method comprising:
identifying said mammal as having a mannosidase alpha class 1A member 2 (MAN1A2) variant within a biological sample from said mammal, and
administering an alternative treatment for non-Hodgkin's lymphoma to said mammal under conditions wherein the severity of a symptom of non-Hodgkin's lymphoma is reduced.

10. The method of claim 9, wherein said mammal is a human.

11. The method of claim 9, wherein said non-Hodgkin's lymphoma is diffuse large B-cell lymphoma.

12. The method of claim 9, wherein said MAN1A2 variant is a single nucleotide polymorphism (SNP).

13. The method of claim 12, wherein said SNP is selected from the group consisting of a rs1289877 SNP, a rs1934249 SNP, a rs907662 SNP, a rs1289869 SNP, a rs1289871 SNP, a rs10801955 SNP, a rs1014516 SNP, a rs1774858 SNP, a rs1707115 SNP, a rs1289855 SNP, a rs1289876 SNP, a rs1289877 SNP, a rs1289878 SNP, a rs1289879 SNP, a rs1289880 SNP, a rs946520 SNP, a rs28648666 SNP, a rs1146297 SNP, a rs1289951 SNP, a rs1289954 SNP, a rs1146298 SNP, a rs1146299 SNP, a rs1289820 SNP, a rs1289960 SNP, a rs75814462 SNP, a rs1289961 SNP, a rs1009549 SNP, a rs1679821 SNP, a rs1289957 SNP, a rs17037239 SNP, a rs1289959 SNP, a rs147796517 SNP, a rs1316011 SNP, a rs2256057 SNP, a rs1289938 SNP, a rs1289939 SNP, a rs884748 SNP, a rs2359248 SNP, a rs77844631 SNP, a rs1289947 SNP, a rs6686707 SNP, a rs1289948 SNP, a rs1315646 SNP, a rs9727433 SNP, a rs6676578 SNP, a rs78876272 SNP, a rs7539143 SNP, a rs10923293 SNP, a rs10923294 SNP, a rs10923297 SNP, a rs78742938 SNP, a rs3767797 SNP, a rs4360510 SNP, a rs7514323 SNP, a rs3920835 SNP, a rs3820500 SNP, a rs2211130 SNP, a rs34368125 SNP, a rs10923316 SNP, a rs1290539 SNP, a rs1290541 SNP, a rs1290542 SNP, a rs1290543 SNP, a rs7546624 SNP, a rs1934249 SNP, a rs2790033 SNP, a rs1290549 SNP, a rs1290551 SNP, a rs1290552 SNP, a rs1290553 SNP, a rs1290556 SNP, a rs1290557 SNP, a rs1290558 SNP, a rs2767318 SNP, a rs1053902 SNP, a rs6667122 SNP, a rs2767320 SNP, a rs2622362 SNP, a rs2622363 SNP, and a rs12738484 SNP.

14. The method of claim 9, wherein said biological sample comprises a fluid sample.

15. The method of claim 14, wherein said fluid sample is blood.

16. The method of claim 9, wherein said alternative treatment for non-Hodgkin's lymphoma comprises a non-rituximab therapeutic agent.

17. The method of claim 16, wherein said non-rituximab therapeutic agent is selected from the group consisting of cyclophosphamide, aldophosphamide, doxorubicin, vincristine, prednisone, a non-rituximab anti-CD20 antibody, and an anti-CD22 antibody.

18. The method of claim 17, wherein said non-rituximab anti-CD20 antibody is ublituximab.

19. The method of claim 17, wherein said anti-CD22 antibody is epratuzumab.

20. The method of claim 9, wherein said alternative treatment for non-Hodgkin's lymphoma comprises a non-chemotherapeutic treatment.

21. The method of claim 20, wherein said non-chemotherapeutic treatment is selected from the group consisting of radiation therapy, immunotherapy, and a stem cell transplant.
